(19) United States
(12) Patent Application Publication
(10) Pub. No.: US 2011/0137395 A1
(43) Pub. Date: Jun. 9, 2011

(54) CATHETER SYSTEM

(75) Inventor: Amir Fargahi, Buelach (CH)

(73) Assignee: BIOTRONIK VI PATENT AG, Baar (CH)

(21) Appl. No.: 12/952,054

(22) Filed: Nov. 22, 2010

Related U.S. Application Data
(60) Provisional application No. 61/267,116, filed on Dec. 7, 2009.

(51) Int. Cl. A61F 2/84; A61M 25/10

(52) U.S. Cl. 623/1.11; 604/96.01

(57) ABSTRACT

A catheter system, comprising a catheter having a distal end and a proximal end; a functional section situated close to the distal end of the catheter; an inner shaft having a guidewire lumen; an outer tubing, such that the outer tubing completely or partially surrounds the functional section; a guidewire outlet having a distal portion and a proximal portion, such that the guidewire outlet passes through the inner shaft and the outer tubing; a guidewire, such that the guidewire is situated on the distal end of the catheter inside the guidewire lumen and is guided out of the catheter through the guidewire outlet, such that the guidewire outlet is attached close to the distal end of the catheter and proximally of the functional section; a cutting device for severing the outer tubing in retraction of same is attached to the distal portion of the guidewire outlet.

### CROSS REFERENCE TO RELATED APPLICATIONS

The invention claims benefit of priority to U.S. patent application Ser. No. 61/267,116 filed on Dec. 7, 2009; the contents of which are herein incorporated by reference in their entirety.

### TECHNICAL FIELD

The invention relates to a catheter system having an inner shaft which surrounds a guidewire lumen and an outer tubing, such that the outer tubing has a functional section.

### BACKGROUND

Catheters, in particular wire-guided catheters, are widely used, primarily in medical applications and procedures. Wire-guided catheters are used in angioplasty and in the implantation of stents in particular. In such applications, the catheter is positioned with the help of a guidewire inserted previously into the patient's body. The distal tip of a catheter guidewire is usually designed to be very flexible, so that it can be bent and rotated to be advanced to the desired location in the patient's body, e.g., within a blood vessel. A wide variety of types of wire-guided catheters are known, e.g., interventional catheters, balloon catheters and catheters for application of self-expanding stents.

With so-called monorail catheters, which are also known as rapid-exchange catheters, the guidewire does not run along the entire length in the guidewire lumen of the catheter, in contrast with over-the-wire catheters, but instead is guided out of the guidewire lumen close to the distal end of the catheter. To this end, the catheter has a guidewire outlet in the appropriate location. Corresponding arrangements are described in U.S. Pat. No. 5,290,241, U.S. Pat. No. 5,324,269 and U.S. Pat. No. 6,251,084, for example.

An arrangement known in the prior art consists of a catheter system having a catheter and a guidewire. The catheter has a guidewire outlet for the guidewire to emerge from the catheter guidewire lumen close to its distal end. Proximally from the guidewire outlet, the outer shaft of the catheter has a type of groove or recess. This groove or recess serves to receive the guidewire in retraction of the outer tubing. When the outer tubing is retracted, it presses on the catheter while at the same time clamping the guidewire, so space must be created for the guidewire.

To produce a catheter system that is as flexible as possible, having a small diameter and therefore being gentle for the patient, the catheter and the outer tubing are manufactured with the smallest possible diameter. This necessarily creates increased friction with movements of the two against one another. A further increase in the friction effect is induced when the guidewire is clamped between the outer tubing and the catheter—as is the case with traditional approaches. This friction is further increased because the guidewire is made of a different material than the catheter and the outer tubing anyway.

The approaches known previously also have the disadvantage that in the case of torsion or bending of the catheter in the patient's body, the process of release of the stent is disturbed or even prevented. In the case of torsion of the catheter, the groove for receiving the guidewire is also subject to torsion when the outer tubing is retracted. As a result, the guidewire does not lie in the groove provided for it but instead it extends beyond the edges and comes to lie in the nonrecessed area of the catheter. The guidewire therefore becomes clamped between the catheter and the outer tubing and makes it difficult or even impossible to retract the outer tubing. The stent cannot be released.

Another disadvantage of the groove design known in the past is that it makes the catheter stiff. In the originally round cross-section of a catheter, a recess is created over the entire length of the catheter by means of a groove. Such a change in shape results in a greater stiffness of the catheter. Due to the greater stiffness and the associated lower flexibility, handling is difficult for the treating physician and may lead to complications, under which the patient suffers.

The object of the present invention is thus to make available a catheter system which eliminates or at least reduces the aforementioned disadvantages. In particular, the friction between the guidewire and the outer tubing in retraction of same, i.e., in the so-called pullback movement, is to be reduced.

### SUMMARY OF THE INVENTION

This object is achieved according to the invention by the features of the independent claim. Advantageous embodiments and advantages of the invention are derived from the additional claims and the description.

The invention is directed to a catheter system, consisting of a catheter, a functional section, an inner shaft with a guidewire lumen, an outer tubing and a guidewire outlet and a guidewire. The guidewire outlet is provided with a cutting device for severing the outer tubing on retraction of same.

By retracting the outer tubing, the functional section is exposed. Friction between the guidewire and the outer tubing in retraction of same, i.e., in the so-called pullback movement, is reduced due to the fact that the outer tubing is severed in the process. Due to the severing of the outer tubing, space is created for the guidewire, which does not come to lie between the catheter and the outer tubing in retraction of the outer tubing but instead continues to lie outside of the catheter.

In addition, possible complications due to torsion of the catheter are avoided by severing the outer tubing at the guidewire outlet. Due to the fact that the guidewire is outside of the catheter during and after retraction of the outer tubing, it remains unimpaired by torsion of the catheter and does not interfere with retraction of the outer tubing itself even if there is torsion of the catheter.

With the inventive catheter system, there is still no stiffening of the catheter due to the design. Due to the fact that the outer tubing is severed and thus creates room for the guidewire in retraction of the outer tubing outside of the catheter, no groove or other recess in the catheter, which would have resulted in a design-related stiffening of the catheter, is necessary.

The catheter has a distal end and a proximal end. The guidewire lumen extends axially through the catheter over its entire length, and is surrounded by the inner shaft. A guidewire outlet is situated close to the distal end of the catheter and proximally of the functional section. The guidewire, which is freely movable up to and through the catheter tip, is situated at the distal end of the catheter within the guidewire lumen and is guided out of the catheter through the guidewire outlet. The guidewire outlet consists of a perforation passing continuously through the inner shaft and the outer tubing. It has a distal portion and a proximal portion. According to the invention, a cutting device for severing the outer tubing is provided on the distal portion of the guidewire outlet.

The materials for the outer tubing and the inner shaft can be derived from the prior art. The outer tubing may thus consist of a plastic (e.g., PA, PEBA, PUR, Teflon), for example, or may be a multilayer plastic. In the case of a multilayer design, the outer layer consists of solid materials such as PA, PEBA, PUR and the inner layer consists of low-friction materials such as HDPE, Teflon. In addition, the outer tubing may have a middle layer which serves either as an adhesion promoter or as a reinforcement. For example, a metal wire, a metal coil or a metal braid is available as reinforcement.

At the site of treatment, the outer tubing, which partially or completely encloses the functional section during the implantation process, is retracted and exposes the functional section. In retraction of the outer tubing, it reaches the cutting device on the distal portion of the guidewire outlet and is severed there. This forms a gap in the outer tubing, which leaves space for the guidewire.

The cutting device, preferably embodied as a blade, may be made of one or more metals, e.g., stainless steel, Co—Cr alloys, Nitinol alloys, etc. or one or more ceramic materials, e.g., zirconate.

The guidewire outlet and the cutting device may be attached to the catheter separately from one another or embodied as a cohesive component. Materials for both variants would include, for example, metals, e.g., stainless steel, a Co—Cr alloy or a Nitinol alloy. However, both may be cast as an injection-molded part, consisting of materials such as polycarbonate, polystyrenes, ABS or K-Resin. The material may also be a thermoplastic material such as PA, PEBA, PUR or silicone.

The functional section on the distal end of the catheter may be designed as a stent-receiving section or as a balloon section, for example.

In a preferred embodiment, the functional section is designed as a stent-receiving section. Stents are currently widely used because they alloy simple and inexpensive treatment of vascular diseases. They often have a tubular or hollow cylindrical basic mesh, which is open on both longitudinal ends. The basic mesh of such an endoprosthesis is inserted by means of a catheter into the body cavity to be treated and serves to support the body cavity, after removal of the catheter. Due to the use of stents, constricted areas in the vessels can be expanded permanently or at least for a certain period of time, resulting in a gain in lumen in the body cavity.

The stent which is provided for implantation is attached to the functional section of the catheter. The stent may be any stent known from the prior art. The stent may be coated with a drug or uncoated. It may be a self-expanding or balloon-dilatable variant. It may also be a permanent stent or a biodegradable stent.

Intraluminal endoprostheses today are often provided with active pharmaceutical substances which are released in the body over a certain period of time. These active pharmaceutical substances may serve to prevent restenoses or agglomerations, for example. Due to the release of active pharmaceutical substances with which such intraluminal endoprostheses are provided, it is possible to perform local treatment alone, i.e., eluting of the drug essentially only into the tissue surrounding the intraluminal endoprosthesis. This process is known as “local drug delivery” (LDD). The site of treatment, where the drug should manifest its pharmacological effect thus borders directly on the site of implantation of the intraluminal endoprosthesis.

The term “active pharmaceutical substance” (or active therapeutic substance or active agent) in the sense of the present invention is understood to be a vegetable, animal or synthetic active ingredient (drug, medication) or a hormone which is used in a suitable dosage as a therapeutic agent to influence states or functions of the body, as a substitute for active ingredients produced naturally by the human or animal body such as insulin and to eliminate disease pathogens, tumors, cancer cells or exogenous substances or to render them harmless. Release of the substance in the environment of the endoprosthesis has a positive effect on the course of healing or counteracts pathological changes in the tissue due to the surgical procedure and/or serves to render malignant cells harmless in oncology.

Such active pharmaceutical substances have an anti-inflammatory and/or anti-proliferative and/or spasmolytic effect, for example, so that restenoses, inflammations or (vascular) spasms can be prevented. In especially preferred exemplary embodiments, such substances may consist of one or more substances in the group of active ingredients including calcium channel blockers, lipid regulators (e.g., fibrates), immunosuppressants, calcineurin inhibitors (e.g., tacrolimus), antiphlogistics (e.g., cortisone or diclofenac), anti-inflammatories (e.g., imidazoles), antiallergics, oligonucleotides (e.g., dODN), estrogens (e.g., genistein), endothelializers (e.g., fibrin), steroids, proteins, hormones, insulins, cytostatics, peptides, vasodilators (e.g., sartans) and antiproliferative substances such as paclitaxel or sirolimus or derivatives thereof.

The stent may be provided with function elements, which have a different composition in at least a portion of their volume in comparison with the material of the basic mesh. These function elements serve to determine the position of the stent in the body or the release of medications, for example.

The position of a stent is often determined by imaging methods, e.g., by means of an X-ray apparatus. The materials used for the basic mesh of such stents usually absorb and/or scatter X-ray radiation only to a slight extent, i.e., they are radiolucent, so the stents are often provided with so-called X-ray markers, which contain a material having a higher absorption of X-rays (radiopaque material).

A self-expanding stent is especially preferred. It may be produced from either a shape memory alloy such as Nitinol or it may have a self-expanding stent design (e.g., a wall stent and/or a coil design). The outer layer here has the task of keeping the stent in its compressed form. In addition, the outer tubing can protect any medication that might be present on the stent from being released during implantation (e.g., due to abrasion or diffusion). On implantation of a self-expanding stent, only the retraction of the outer tubing is sufficient and the stent returns to its originally imposed dilated position. It then conforms to the vascular wall and presses against the stenosis, so that the location to be treated is reopened or expanded and the flow of body fluid in the vessel is no longer hindered at all or not to the previous extent. When the outer tubing is retracted, it is severed with the help of the cutting device.

In another preferred embodiment, the stent is balloon-dilatable. The outer tubing here may protect any medication that is present, having been applied to the stent (coating), or may hold the stent more securely and undisplaceably on the catheter. The outer tubing may partially or completely surround the stent. As soon as the outer tubing is retracted, it is severed with the help of the cutting device. A balloon is attached to the catheter beneath the stent. If the balloon is dilated—by introducing a liquid under pressure—the stent on top of it expands until it has reached the desired expanded shape. When the liquid is drained out, the balloon collapses again and may then be removed. The stent remains behind at the treatment site.

Intraluminal endoprostheses made of a biodegradable material are also currently in use. Biodegradation here is understood to refer to hydrolytic, enzymatic or metabolic degradation processes in the living organism caused mainly by the body fluids coming in contact with the endoprosthesis and leading to a gradual dissolution of at least large portions of the endoprosthesis. The term biocorrosion is frequently used as synonymous with the term biodegradation. The term bioresorption comprises the subsequent resorption of the degradation products by the living organism. Such biodegradable materials may be implemented from polymers or metals. In conjunction with stents, the abbreviation AMS (absorbable metal stent) is customary. Such stents contain a biodegradable metal, preferably magnesium and/or a magnesium alloy.

The functional section designed as a stent-receiving section may hold one or more stents. For implantation of multiple stents during a catheter procedure, this system may be designed so that the outer tubing is retracted only until a stent is released, beginning with the stent in the farthest distal position. Next, the catheter is moved to the next site to be treated, where the next stent is released by further retraction of the outer tubing, etc. The outer tubing is cut to the part required for release of the stent.

In another preferred embodiment, the functional section is designed as a balloon section. The balloon attached to the balloon section is dilated at the site of treatment and thus widens the constricted blood vessel. At the same time, it has the task of dispensing a medication at the treatment site (drug-coated balloon, drug-eluting balloon). In the time during which the balloon is in dilated faun in the blood vessel, it comes in contact with the vascular walls and dispenses one or more medications to the vascular wall. An outer tubing drawn over the balloon serves to protect the medication from being released (e.g., by abrasion or diffusion) during its path through the vascular system to the treatment site. At the treatment site, the outer tubing is retracted and is severed in the process. Next the balloon is dilated, thus dispensing the medication at the same time. After compressing the balloon, it can be retracted out of the vessel together with the catheter system.

In another preferred embodiment, a protective device is attached over the cutting device. This protective device serves to protect the tissue and in particular the vascular walls during the entire procedure. Since the cutting device is naturally polished to a sharp edge, there is the risk of injury of the tissue with even the slightest contact with tissue. The purpose is of course to completely prevent the operator from being able to freely execute the required movement of the catheter system to reach the treatment site.

The protective device is preferably designed as a half-shell, which covers only the cutting device, or as a tube completely surrounding the catheter at the location where the cutting device is attached.

### DESCRIPTION OF THE DRAWINGS

The invention will now be explained in greater detail on the basis of exemplary embodiments in conjunction with the figures.

FIG. 1 shows schematically the catheter system, in which the functional section is designed as a stent-receiving section having a self-expanding stent.

FIG. 2 shows schematically the catheter system, in which the functional section is designed as a stent-receiving section having a balloon-dilatable stent.

FIG. 3 shows schematically the catheter system, in which the functional section is designed as a balloon section.

FIG. 4 shows schematically the cutting device.

FIG. 5 shows schematically the cutting device in combination with the guidewire outlet.

FIG. 6 shows schematically the protective device in the form of a half-shell.

FIG. 7 shows schematically the protective device in the form of a tube.

### DETAILED DESCRIPTION

The figures are schematic diagrams of the invention. They illustrate nonspecific parameters of the invention. In addition, the figures merely represent typical embodiments of the invention and should not restrict the invention to the embodiments shown here.

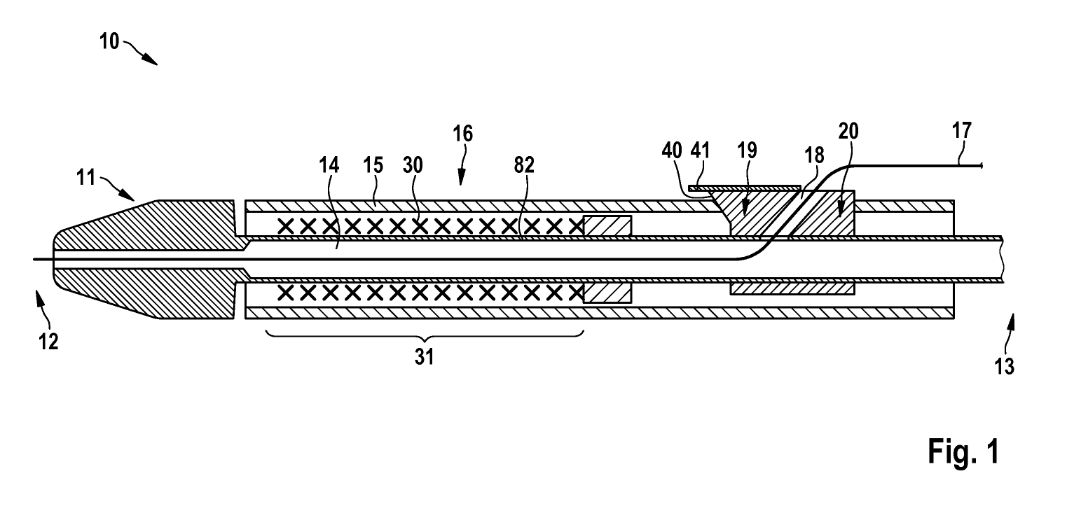

FIG. 1 shows a preferred embodiment of the catheter system 10 for inserting a self-expanding stent 30. The catheter system 10 includes a catheter 11 with a distal end 12 and a proximal end 13. The catheter 11 has an inner shaft 82 with a guidewire lumen 14 and a guidewire outlet 18. The guidewire 17 runs inside the guidewire lumen 14 on the distal end of the catheter 12. The guidewire 17 leaves the guidewire lumen 14 through the guidewire outlet 18 and continues to run outside of the catheter 11.

Furthermore, the catheter system 10 includes a functional section 31, which serves to receive the self-expanding stent 30 near the distal end of the catheter 12. For the purpose of implantation, a crimped self-expanding stent 30 is attached over the functional section 31. By crimping, it is compressed down to the smallest possible diameter and therefore has the small cross section required for implantation. At the treatment site, the self-expanding stent 30 is dilated and released from the functional section 31 and thus from the entire catheter.

The self-expanding stent 30 is completely or partially surrounded by an outer tubing 15. With self-expanding stents, the outer tubing 15 serves to retain the crimped state of the stent 30 during implantation. At the treatment site, the outer tubing 15 is retracted and the stent is expanded and then assumes its dilated state imposed previously.

The guidewire outlet 18 is close to the distal end of the catheter 12 and is proximal of the functional section 31, the stent-receiving section here. The guidewire outlet 18 describes an area on the catheter having a continuous perforation in its center through the inner shaft 82 and the outside tubing 15. The guidewire outlet 18 has a distal portion 19 and a proximal portion 20.

The inventive cutting device 40 is provided on the distal portion 19 of the guidewire outlet 18. It is attached to the inner shaft 82 and protrudes radially outward. After reaching the implantation site, the self-expanding stent 30 is released by retracting the outer tubing 15. Simultaneously with this retraction, the outer tubing 15 reaches the cutting device 40 and is severed.

Since the outer tubing 15 consists of a relatively rigid material, it leaves enough room for the guidewire 17 but it is so rigid that it remains in a tubular shape and can be drawn gently out of the body along with the catheter 11.

In addition, FIG. 1 shows schematically the protective device 41, which shields the cutting device 40 with respect to the environment, in particular the vascular walls.

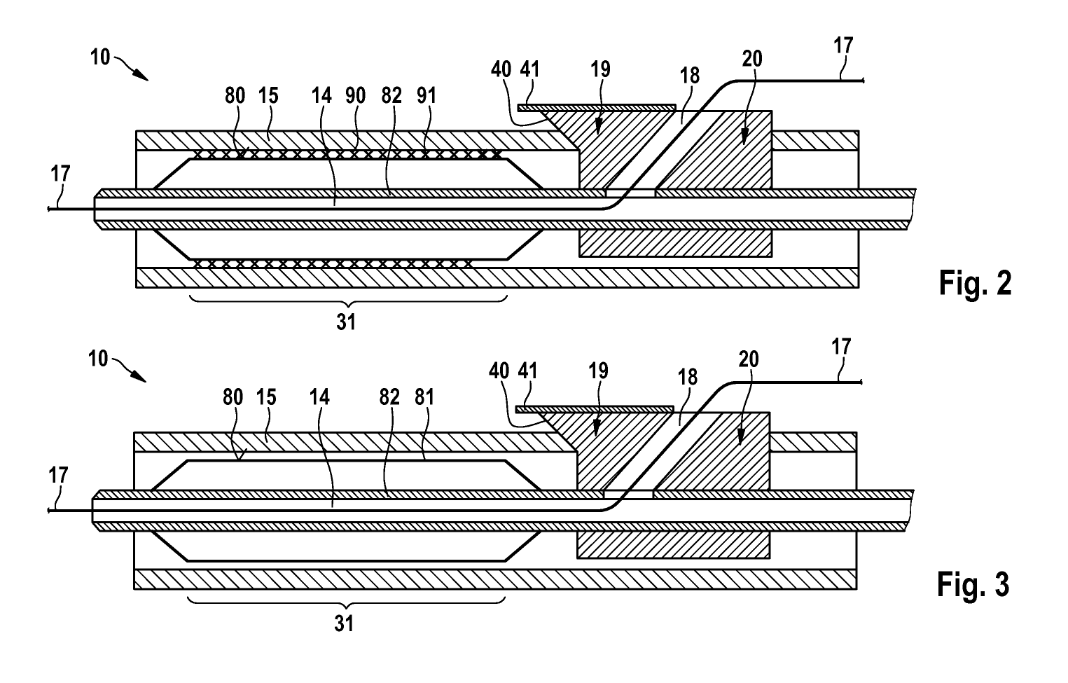

FIG. 2 shows another preferred embodiment of the catheter system 10. The catheter 11 serves to implant a balloon-dilatable stent 90. The design is similar to the design described in conjunction with FIG. 1. In addition, the catheter system shown in FIG. 2 has a balloon 91, which serves to dilate the stent 90 at the treatment site. It is attached to the outer tubing 15 and beneath the balloon-dilatable stent 90.

By analogy with FIG. 1, FIG. 2 shows this additional preferred embodiment of the catheter system 10 for insertion of a balloon-dilatable stent 90. The catheter system 10 includes a catheter 11 having a distal end 12 and a proximal end 13. The catheter 11 has an inner shaft 82 with a guidewire lumen 14 and a guidewire outlet 18. The guidewire 17 runs within the guidewire lumen 14 on the distal end of the catheter 12. The guidewire 17 leaves the guidewire lumen 14 through the guidewire outlet 18 and continues to run outside of the catheter 11.

Furthermore, the catheter system 10 includes a functional section 31, which serves to receive the balloon-dilatable stent 90 close to the distal end of the catheter 12. A balloon 91 is attached over the functional section 31. For the purpose of implantation, a crimped balloon-dilatable stent is attached to this balloon. Due to the crimping, it is compressed to the smallest possible diameter and therefore has the cross section required for implantation. At the treatment site, the balloon-dilatable stent 90 is dilated with the help of the balloon 91 and is released from the functional section 31 and thus from the entire catheter.

The balloon-dilatable stent 90 is completely or partially surrounded by an outer tubing 15. The outer tubing 15 protects a medication that might be present on the stent surface or serves to secure the stent on the balloon 91 and/or on the catheter 10. The outer tubing 15 is retracted, the balloon 91 is dilated and thus the stent is expanded at the treatment site.

The guidewire outlet 18 is situated closed to the distal end of the catheter 12 and proximally from the functional section 31, here the stent-receiving section. The guidewire outlet 18 describes an area on the catheter, which has a continuous perforation through the inner shaft 82 and the outside shaft 15. The guidewire outlet 18 has a distal portion 19 and a proximal portion 20.

The inventive cutting device 40 is provided on the distal portion 19 of the guidewire outlet 18. It is attached to the inner shaft 82 and protrudes radially outward. After reaching the treatment site, the outer tubing 15 is retracted and therefore reaches the cutting device 40 and is severed.

Since the outer tubing 15 consists of a relatively rigid material, it leaves enough space for the guidewire 17 but is rigid enough so that it remains in a tubular shape and can be retracted gently from the body along with the catheter 11.

In addition, FIG. 2 shows schematically the protective device 41 which shields the cutting device 40 with respect to the environment, in particular the vascular walls.

FIG. 3 shows another preferred embodiment of the catheter system 10. In FIG. 3 the functional section 31 is designed as a balloon section. A balloon 81 which has a drug coating 80 is attached to the inner shaft 82. The balloon 81 is completely or partially surrounded by an outer tubing 15. The outer tubing 15 serves to protect the medication from being released from the balloon surface.

The inventive cutting device 40 is provided on the distal portion 19 of the guidewire outlet 18. It is attached to the inner shaft 82 and protrudes radially outward. After reaching the treatment site, the outer tubing 15 is retracted and therefore reaches the cutting device 40, where it is severed. The balloon 81 is dilated. As soon as it touches the walls of the vessel, it dispenses the medication to the vascular wall.

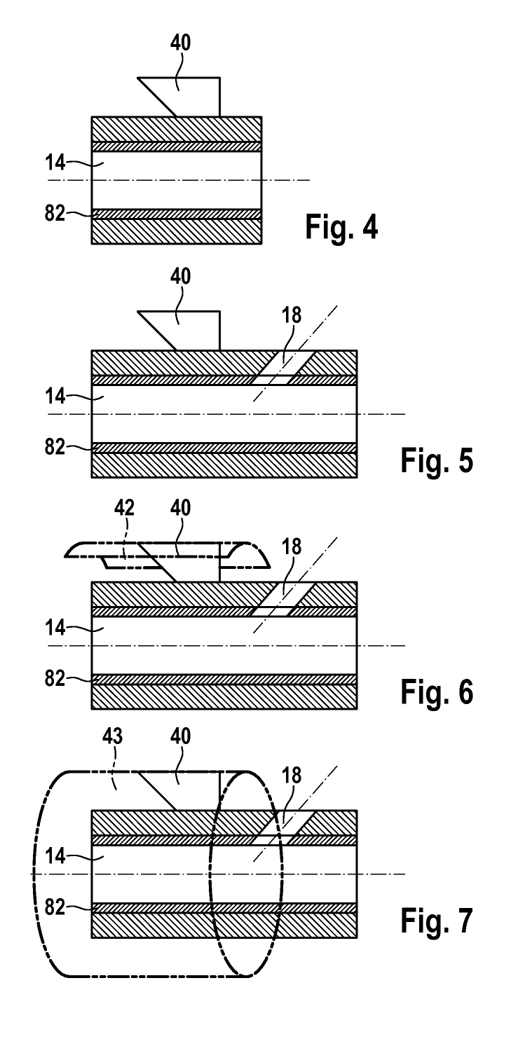

FIG. 4 shows an enlarged detail of the cutting device 40. The cutting device 40 may be arranged on the catheter independently of the guidewire outlet 18. The only prerequisite is that the cutting device 40 must be arranged distally of the guidewire outlet 18.

FIG. 5 shows the combination of cutting device 40 and guidewire outlet 18 in one component. Here the cutting device 40 and the guidewire outlet 18 are relatively close together and are secured by a component.

An enlarged detail of various embodiments of the protective device 41 is shown in FIGS. 6 and 7. FIG. 6 shows a protective device in the form of a half-shell 42, which is attached directly to the cutting device 40 and surrounds it. FIG. 7 shows schematically a protective device in the form of a tube 43. The tube 43 is attached in such a way that it surrounds the complete catheter 11 in the area of the cutting device 40. The catheter 11 runs axially through the tube 43; and the tube 43 has a diameter corresponding exactly to the diameter of the catheter 11 with the cutting device 40.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NUMERALS

10 catheter system
11 catheter
12 distal end of the catheter
13 proximal end of the catheter
14 guidewire lumen
82 inner shaft
15 outer tubing
16 outer area of the catheter
17 guidewire
18 guidewire outlet
19 distal portion of the guidewire outlet
20 proximal portion of the guidewire outlet
30 self-expanding stent
31 functional section
40 cutting device
41 protective device
42 protective device as a half-shell
43 protective device as a tube
80 drug-coated balloon
81 medication coating
90 balloon-dilatable stent
91 balloon

What is claimed is:

1. A catheter system, comprising
a catheter having a distal end and a proximal end;
a functional section situated close to the distal end of the catheter;
an inner shaft having a guidewire lumen;
an outer tubing, whereby the outer tubing completely or partially surrounds the functional section;
a guidewire outlet having a distal portion and a proximal portion, whereby the guidewire outlet passes through the inner shaft and the outer tubing;
a guidewire, the guidewire being situated on the distal end of the catheter inside the guidewire lumen and being guided out of the catheter through the guidewire outlet, the guidewire outlet being attached close to the distal end of the catheter and proximally of the functional section;
characterized in that a cutting device for severing the outer tubing in retraction of same is attached to the distal portion of the guidewire outlet.

2. The catheter system according to claim 1, characterized in that the cutting device is affixed on the inner shaft.

3. The catheter system according to claim 1, characterized in that the cutting device is in the form of a blade.

4. The catheter system according to claim 1, characterized in that a protective device is attached over the cutting device.

5. The catheter system according to claim 4, characterized in that the protective device is in the form of a half-shell.

6. The catheter system according to claim 4, characterized in that the protective device is in the form of a tube.

7. The catheter system according to claim 1, characterized in that the functional section is designed as a stent-receiving section.

8. The catheter system according to claim 1, characterized in that the functional section is designed as a balloon section.

9. The catheter system according to claim 7, characterized in that the stent is self-expandable or balloon-expandable.
